(19) United States
(12) Patent Application Publication
(10) Pub. No.: US 2024/0123143 A1
(43) Pub. Date: Apr. 18, 2024

(54) PARALLEL INFUSION DEVICE, SYSTEM, AND METHOD

(71) Applicant: CareFusion 303, Inc., San Diego, CA (US)

(72) Inventor: Nolan Leendert PUPPING, Carlsbad, CA (US)

(21) Appl. No.: 18/488,904

(22) Filed: Oct. 17, 2023

Related U.S. Application Data
(60) Provisional application No. 63/417,267, filed on Oct. 18, 2022.

(51) Int. Cl. A61M 5/168; A61M 5/142; A61M 5/165

(52) U.S. Cl. CPC A61M 5/16813; A61M 5/14228; A61M 5/165; A61M 2205/3355

(57) ABSTRACT

Infusion devices having multiple pumping segments connected in parallel to a fluid source and configured to operate together to pump a fluid from the fluid source are described herein. An infusion device includes a first deformable body coupled between the fluid source and an IV set and a second deformable body coupled between the fluid source and the IV set in parallel with the first deformable body. The first deformable body is configured to be hand-compressed and direct a portion of fluid from the first deformable body to an outlet of the IV set for administering the fluid to a patient at a first flow rate. The second deformable body is configured to be hand-compressed and direct another portion of fluid from the second deformable body to the outlet of the IV set for administering the fluid to the patient at a second flow rate.

### CROSS REFERENCE TO RELATED APPLICATION PATENT

This application claims priority under 35 U.S.C. § 119 to U.S. Provisional Application No. 63/417,267, entitled “AN INFUSION DEVICE HAVING MULTIPLE PUMPING SEGMENTS CONNECTED TO A COMMON FLUID SOURCE AND CONFIGURED TO OPERATE IN PARALLEL WITH EACH OTHER TO PUMP A FLUID FROM THE COMMON FLUID SOURCE”, filed Oct. 18, 2022, the disclosure of which is incorporated herein by reference in its entirety.

### FIELD OF THE INVENTION

The present disclosure generally relates to medical fluid distribution systems, and, in particular, to a plurality of hand pumps for distributing a medical fluid from an IV bag.

### BACKGROUND

Medical treatments often include the infusion of a medical fluid (e.g., a saline solution or a liquid medication) to patients using an infusion pump that distributes the medical fluid from an intravenous (IV) bag to a patient. When an infusion pump is unavailable, medical practitioners use their hands to compress or otherwise squeeze on the IV bag to administer the medical fluid. Manually compressing the IV bag can result in hand fatigue which greatly inconveniences medical practitioners.

As such, there is a need for devices and methods of use thereof that can distribute medical fluids from an intravenous (IV) bag to a patient without fatiguing a medical practitioner.

### SUMMARY

The disclosed subject matter relates to a plurality of hand pumps for use with IV sets. In certain implementations, the plurality of hand pumps for use with an IV set is disclosed that includes at least two deformable bodies defining an inlet, an outlet, and a deformable body volume in fluid communication with the inlet and the outlet. Each deformable body can be compressed to increase a flow rate of fluid in the IV set.

In accordance with some implementations, an infusion device having multiple pumping segments connected to a common fluid source and configured to operate in parallel with each other to pump a fluid from the common fluid source is disclosed. The infusion device includes a first deformable body fluidically coupled between the fluid source and an IV set, the first deformable body configured to be hand-compressed by a user and, when compressed, direct a first portion of fluid from the fluid source from the first deformable body to an outlet of the IV set for administering the fluid to a patient at a first flow rate. The infusion device also includes a second deformable body fluidically coupled between the fluid source and the IV set in parallel with the first deformable body, the second deformable body configured to be hand-compressed by a user and, when compressed, direct a second portion of fluid from the fluid source from the second deformable body to the outlet of the IV set for administering the fluid to the patient at a second flow rate. The multiple pumping segments are configured to, when compressed according to respective timing patterns offset from each other, deliver the first portion of fluid from the fluid source from the first pumping segment to the IV set while filling the second pumping segment with the second portion of fluid from the fluid source from the fluid source, and to deliver the second portion of fluid from the fluid source from the second pumping segment to the IV set while the first pumping segment is filled from the fluid. Examples of the plurality of hand pumps are described below in reference to FIGS. 3A-4C.

In some implementations, the first deformable body is fluidically coupled between the fluid source and the IV set via a first inlet and the second deformable body is fluidically coupled between the fluid source and the IV set via a second inlet. In some implementations, the first and/or second deformable body are simultaneously compressed. Alternatively, in some implementations, the first and/or second deformable body are compressed in an alternating sequence. In some implementations, the first and second flow rates are the same.

In some implementations, the infusion device further includes one or more filters fluidically coupled to the first and/or second deformable body. The one or more filters capture particulate from the respective fluid directed from the first and/or second deformable body. In some implementations, the first and/or second deformable body, when compressed, direct back flow from the first and/or second deformable body through the one or more filters, agitating particulate captured in the filter. In some implementations, the one or more filters are disposed adjacent to a first and/or second inlet of the first and second deformable bodies, respectively. In some implementations, the one or more filters are disposed within the first and/or second inlet of the first and second deformable bodies, respectively. Examples of the one or more filters are described below in reference to FIGS. 3A-4C.

In some implementations, the first and second deformable bodies include a resilient material such that the first and second deformable bodies rebound to an uncompressed state. In some implementations, the first and second deformable bodies include a transparent material or a semi-transparent material. In some implementations, the first and second deformable bodies include a substantially cylindrical shape.

In accordance with certain implementations, an infusion system having multiple pumping segments connected to a common fluid source and configured to operate in parallel with each other to pump a fluid from the common fluid source is disclosed. The infusion system includes a first portion of tubing, a second portion of tubing, and multiple pumping segments configured to operate in parallel. The multiple pumping segments include a first deformable body fluidically coupled to the first portion of tubing and a second deformable body fluidically coupled to the first portion of tubing. The first deformable body is configured to be hand-compressed by a user and, when compressed, direct a first portion of fluid from the fluid source from the first deformable body to an outlet fluidically coupled to the second portion of tubing for administering the fluid to a patient at a first flow rate. The second deformable body is configured to be hand-compressed by a user and, when compressed, direct a second portion of fluid from the fluid source from the second deformable body to the outlet fluidically coupled to the second portion of tubing for administering the fluid to the patient at a second flow rate. The multiple pumping segments are configured to, when compressed according to respective timing patterns offset from each other, deliver the first portion of fluid from the fluid source from the first pumping segment to the IV set while filling the second pumping segment with the second portion of fluid from the fluid source from the fluid source, and to deliver the second portion of fluid from the fluid source from the second pumping segment to the IV set while the first pumping segment is filled from the fluid.

In some implementations, the first and/or second deformable body are simultaneously compressed. In some implementations, the first and/or second deformable body are compressed in an alternating sequence. In some implementations, the first and second flow rates are the same. In some implementations, the first and second deformable bodies include a resilient material such that the first and second deformable bodies rebound to an uncompressed state.

In some implementations, the IV set further includes one or more filters fluidically coupled to the first and/or second deformable body. The one or more filters capture particulate from the fluid directed from the first and/or second deformable body.

In accordance with certain implementations, a method of forming an apparatus for accelerating a fluid from a fluid source to a patient is disclosed. The method includes connecting, between a first union connector configured to fluidly connect with an upstream infusion line for receiving the fluid from the fluid source and a second union connector, downstream from the first union connector, configured to connect to a downstream infusion line for providing the fluid to an IV set, a first deformable body configured to be hand-compressed by a user. The first deformable body, when compressed, directs a first portion of the fluid from the first deformable body to the IV set for administering the first portion of the fluid to the patient at a first accelerated flow rate. The method includes connecting, between the first and second union connectors, a second deformable body in parallel with the first deformable body. The second deformable body is configured to be hand-compressed by the user and, when compressed, direct a second portion of the fluid from the second deformable body to the IV set for administering the fluid to the patient at a second accelerated flow rate. The first and second deformable bodies are configured to, when compressed according to respective timing patterns offset from each other, operate together to deliver fluid from the fluid source by delivering the first portion of the fluid from the first pumping segment to the IV set while filling the second pumping segment with the second portion of the fluid from the fluid source, and delivering the second portion of the fluid from the second pumping segment to the IV set while the first pumping segment is filled with the first portion of the fluid from the fluid, such that the fluid is delivered from the fluid source to the patient at a flow rate greater than the first and second default flow rates and a default flow rate of the fluid source.

In some implementations, the method includes connecting, a drip chamber upstream of the first union connector that is configured to provide a visual indicator of the default flow rate of the fluid source.

Note that the various implementations described above can be combined with other implementations described herein (e.g., keep an impedance for a single or group of neuromuscular-signal sensors can be combined with matching of impedances, such that impedances can be both matched and be kept within particular ranges of impedance values). The features and advantages described in the specification are not all inclusive and, in particular, additional features and advantages will be apparent to one of ordinary skill in the art in view of the drawings, specification, and claims. Moreover, it should be noted that the language used in the specification has been principally selected for readability and instructional purposes.

### BRIEF DESCRIPTION OF THE DRAWINGS

So that the present disclosure can be understood in greater detail, a more particular description may be had by reference to the features of various implementations, some of which are illustrated in the appended drawings. The appended drawings, however, merely illustrate pertinent features of the present disclosure. The description may admit to other effective features as the person of skill in this art will appreciate upon reading this disclosure.

FIG. 1 illustrates an IV pump administering a medical fluid to a patient.

FIG. 2 illustrates a conventional IV set 200.

FIGS. 3A-3C illustrates an IV set with a plurality of hand pumps, in accordance with some implementations.

FIGS. 4A-4C illustrate another IV set with a plurality of hand pumps, in accordance with some implementations.

FIG. 5 is a flowchart illustrating a method of forming an apparatus for accelerating a fluid from a fluid source to a patient, in accordance with some implementations.

FIG. 6 is a conceptual diagram illustrating an example electronic system for controlling a pump, according to aspects of the subject technology.

In accordance with common practice, the various features illustrated in the drawings are not drawn to scale. Accordingly, the dimensions of the various features may be arbitrarily expanded or reduced for clarity. In addition, some of the drawings may not depict all of the components of a given system, method, or device. Finally, like reference numerals denote like features throughout the specification and figures.

### DETAILED DESCRIPTION

Numerous details are described herein in order to provide a thorough understanding of the example implementations illustrated in the accompanying drawings. However, some implementations may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the implementations described herein.

The disclosed plurality of hand pumps is configured to fluidically couple with an IV bag and accelerate a fluid from the IV bag to a patient for treatment. In particular, each hand pump of the plurality of hand pumps is configured to deform when compressed (or squeezed). When a hand pump of the plurality of hand pumps is compressed, a flow of fluid from the IV bag to a patient is increased. Additionally, the plurality of hand pumps reduce fatigue experienced by clinicians when compressing hand pumps incorporated in conventional IV sets.

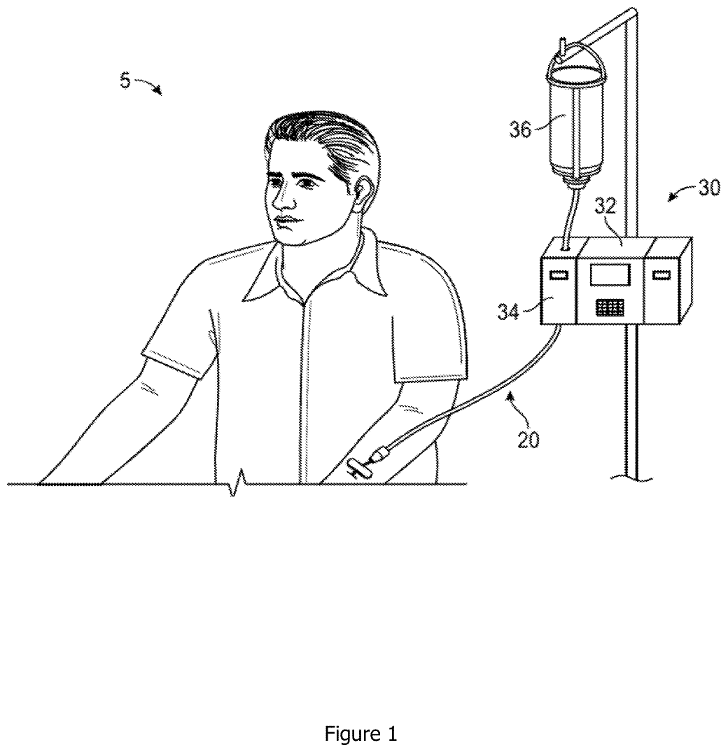

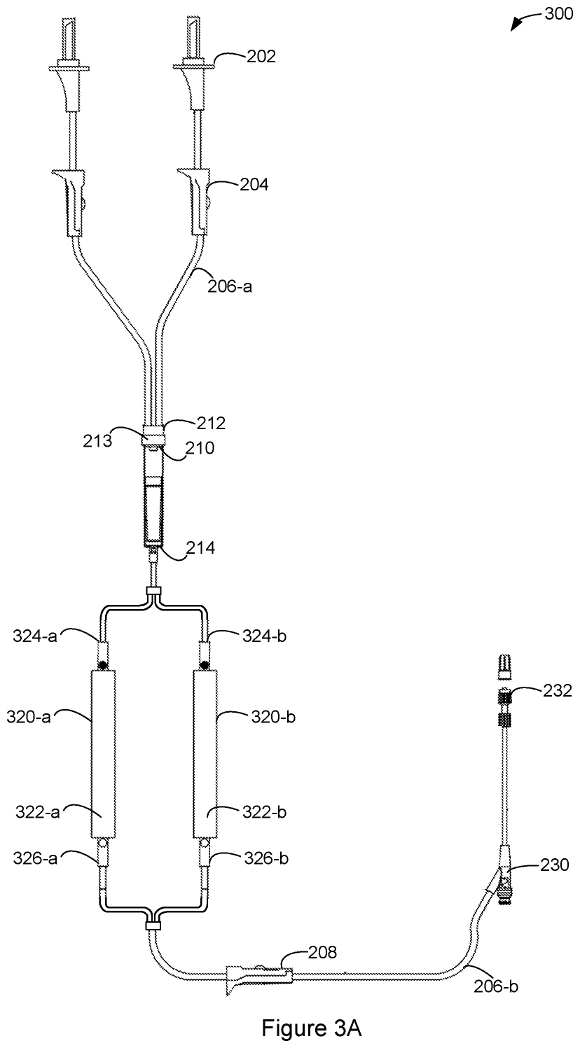

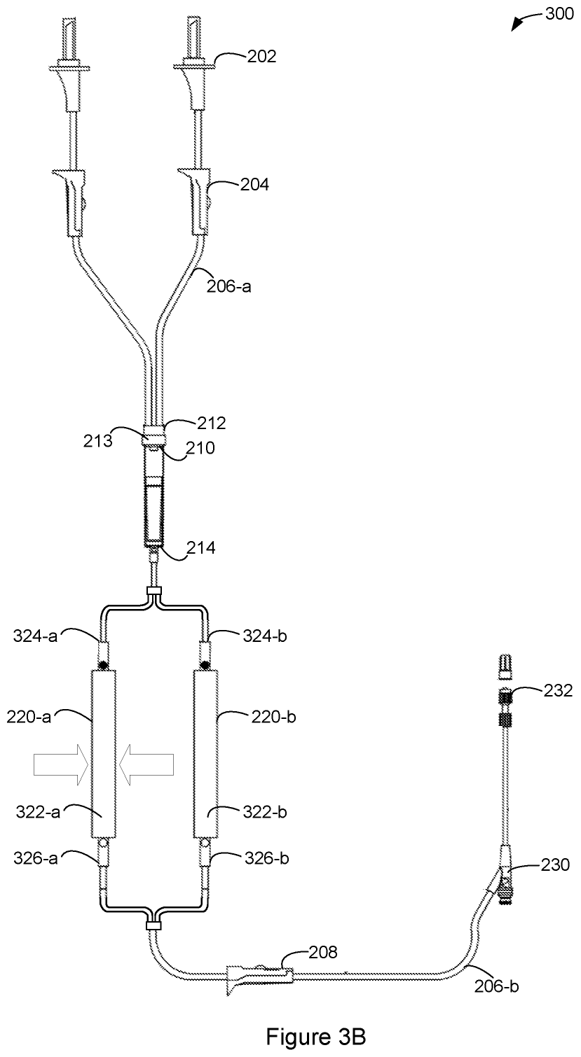

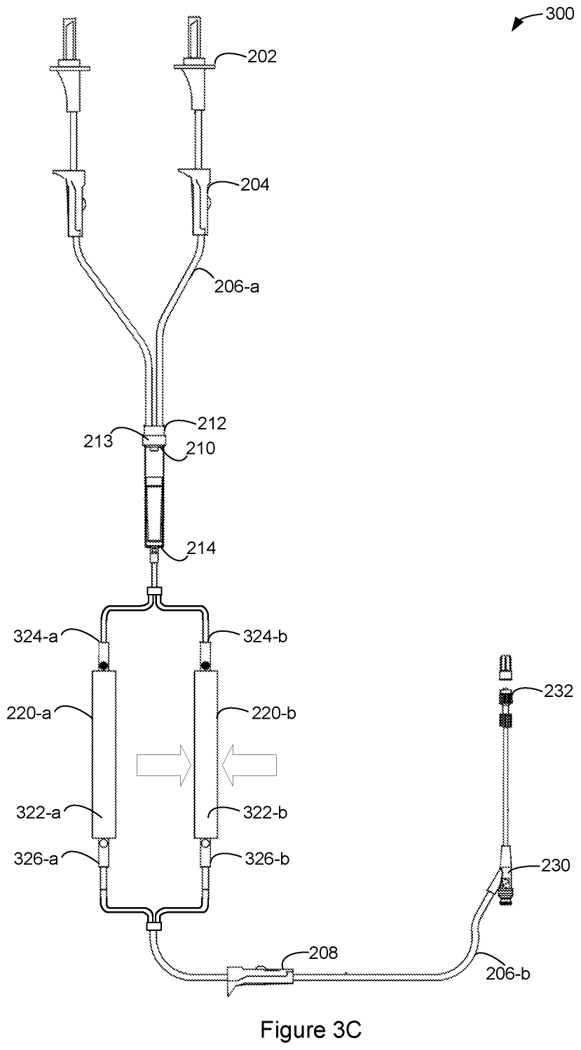

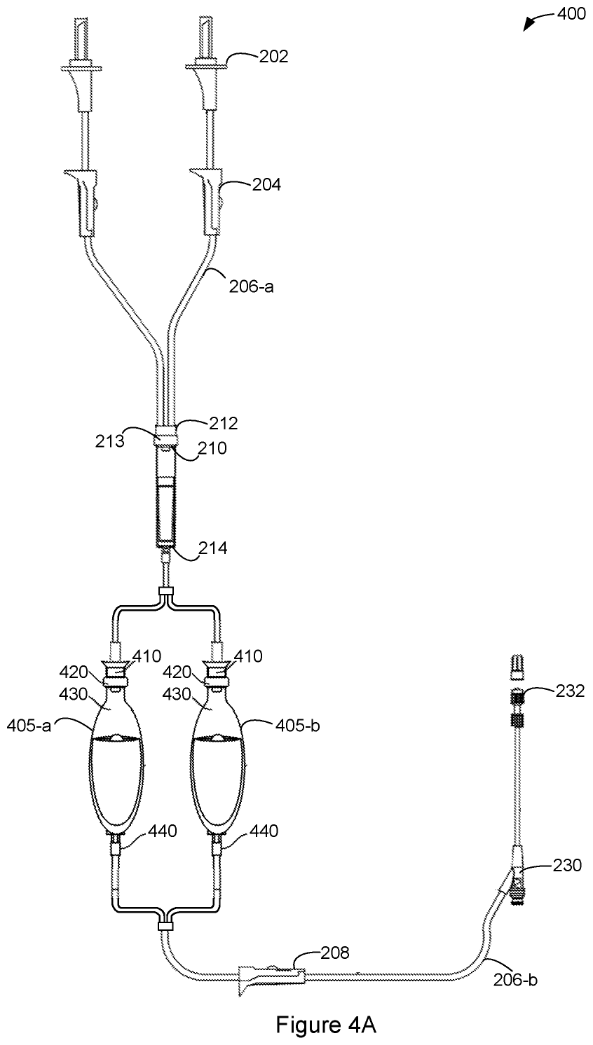

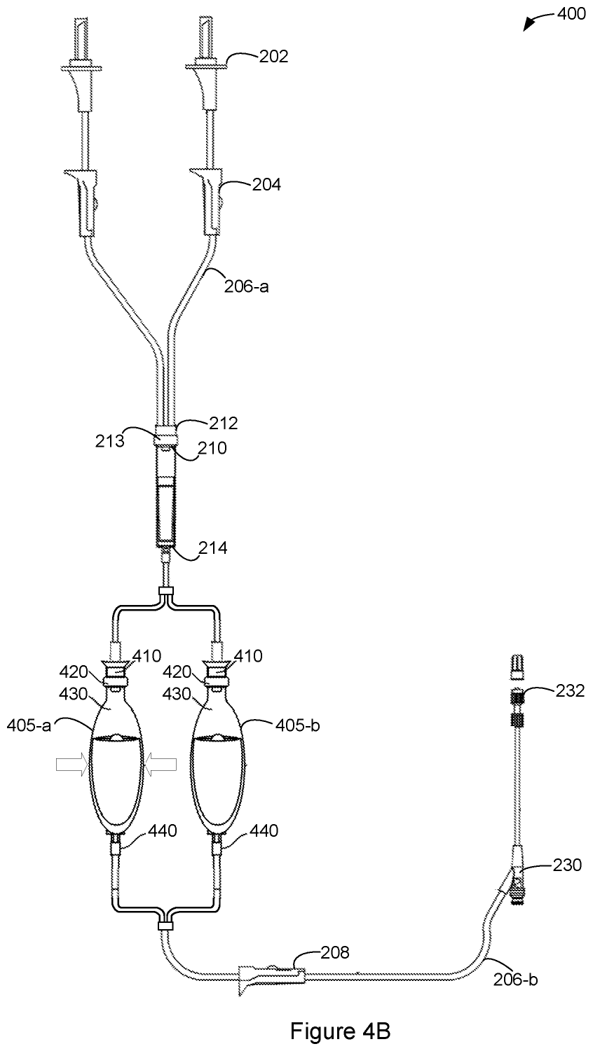

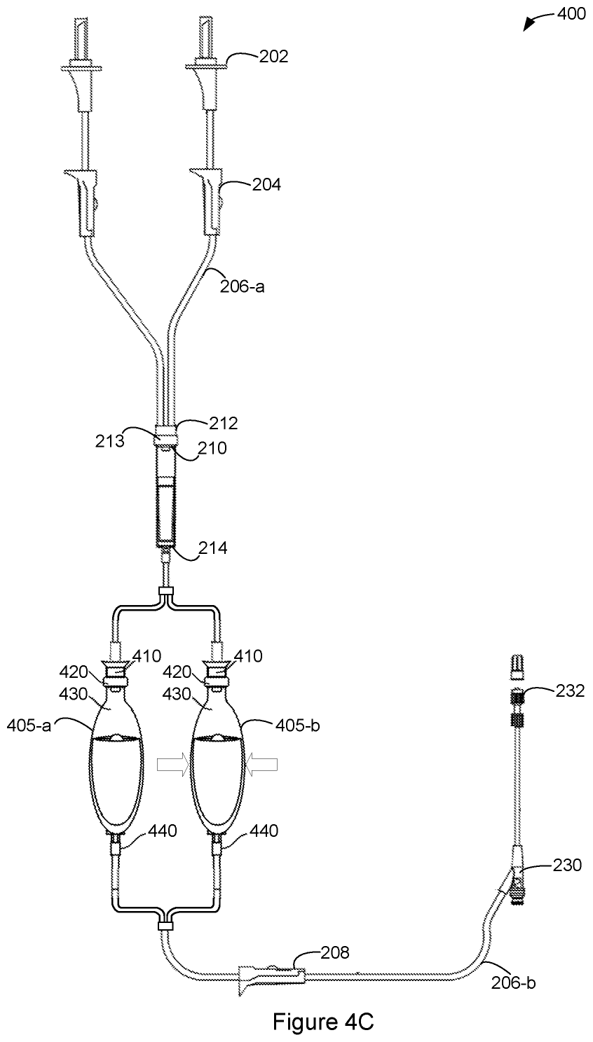

FIG. 1 illustrates an IV pump administering a medical fluid to a patient. The IV pump 30 includes a controller 32 and one or more pump modules 34. An IV set 20 is connected between a container 36 (e.g., an IV bag) of the medical fluid and the patient 5. During operation, IV pump 30 delivers the medical fluid to the patient 5. The IV pump 30 is configured to administer the medical fluid to the patient at a predetermined and uniform rate. In some implementations, the IV set 20 is utilized to further accelerate a flow of medical fluid from the container 36 (or the IV pump 30) to the patient. For example, as shown and described below in reference to FIGS. 3A-4C, the IV set 20 can include a plurality of hand pumps that can be used to further accelerate the flow of medical fluid. In addition, in some implementations, the plurality of hand pumps is used to agitate any particulate trapped within one or more filters (of the IV set 20 and/or the IV pump 30), increasing flow through the filter and the IV set 20. During operation (i.e., the administration of medical fluid to the patient), the filter(s) can be clogged with particulate, reducing flow through the IV set 20. The filter(s) is configured to prevent the transfer of bacteria, microorganisms, and/or other pathogens. The plurality of hand pumps accelerates the flow of medical fluid and/or agitate any particulate trapped within one or more filters via the application of a force to the plurality of hand pumps (e.g., squeezing the plurality of hand pumps) as discussed below in reference to FIGS. 3A-4C.

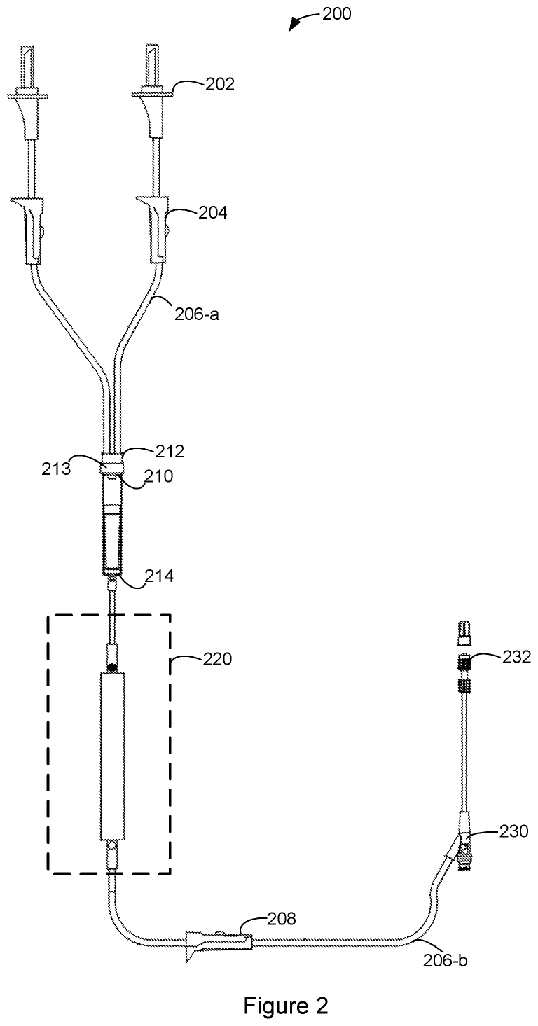

FIG. 2 illustrates a conventional IV set 200. The IV set 200 includes one or more connectors 202, one or more clamps 204 and 208, tubing 206, a drip chamber 210, a hand pump 220, an injection site 230, and an outlet port 232. As described above in reference to FIG. 1, the IV set 200 delivers fluid from a fluid source, such as a container 36, to a patient 5 through tubing 206. More specifically, fluid from the fluid source is introduced into a first portion of the tubing 206-a of the IV set 200 and delivered to the patient 5 (e.g., via a flow generated by an IV pump 30 (FIG. 1) and/or a hand pump 220). The connectors 202 facilitate coupling and/or fluid communication between the first portion of the tubing 206-a and a fluid source (e.g., one or more containers 36). More specifically, the connectors 202 fluidically couple the first portion of the tubing 206-a to one or more one or more containers 36. In some implementations, the connectors 202 can be connector spikes that pierce a membrane of the container 36 to permit fluid communication from the container 36 into the tubing 206. Alternatively, in some implementations, the connectors 202 can be needleless connectors to avoid inadvertent piercing of membranes when fluidically coupling the first portion of the tubing 206-a and the fluid source. In some implementations, the needleless connectors can include a no-drip feature to prevent leaks or surface contamination. In some implementations, the needleless connectors can further include a luer lock to prevent accidental discharges. In some implementations, the needleless connectors engage with fluid containers that include mating connectors.

In some implementations, a second portion of the tubing 206-b is coupled to the patient 5 via the outlet port 232. Additional medical fluids or treatments can be introduced to the patient via the IV set 200. In some implementations, additional medical fluids or treatments can be introduced into the IV set 200 via the injection site 230. In some implementations, the fluid container allows for pulling aliquots of blood for analysis.

Clamps 204 and 208 are configured to control fluid flow through the tubing 206 of the IV set 200. For example, clamps 204 and 208 can occlude the flow of fluid through the tubing 206 such that no fluid is distributed to the patient 5. Alternatively, clamps 204 and 208 can partially occlude the flow of fluid through the tubing 206 such that the flow of fluid distributed to the patient 5 is reduced. The clamps 204 and 208 can be roller clamps, pinch clamps, slide clamps, and/or other clamps known in the art.

In some implementations, the drip chamber 210 is formed of a transparent material or a semi-transparent material to provide a visual indicator of the flow rate of a medical fluid therethrough. The drip chamber 210 is configured to allow clinicians and/or other medical practitioners to monitor and adjust a flow rate of the medical fluid based on the visual indicator provided by the drip chamber 210 (e.g., counting the number of drops per second). During operation, medical fluid can drip or otherwise flow through the chamber volume of the drip chamber 210. Medical fluid can enter the drip chamber 210 through an upper portion or inlet portion 212 defined in the drip chamber 210. The inlet portion 212 is in fluid communication with the first portion of tubing 206-a. Fluid flow exits the drip chamber 210 through a lower portion or outlet portion 214. The outlet portion 214 can be in fluid communication with the second portion of the tubing 206-b. In some implementations, the drip chamber 210 can equalize pressure differentials between the chamber volume and the environment during operation. In some implementations, the drip chamber 210 can be formed from a resilient material to allow the drip chamber 210 to be squeezed or compressed to draw in medical fluid for priming of an IV system. In some implementations, the drip chamber 210 draws in medical fluid for priming the IV system. As can be appreciated, the drip chamber 210 can be filled with a desired volume of medical fluid during the priming operation.

In some implementations, the drip chamber 210 includes a filter for filtering the medical fluid passing therethrough. In some implementations, the filter 213 is disposed within the drip chamber 210. In some implementations, the filter 213 is disposed within the inlet portion 212 or the outlet portion 214 of the drip chamber 210. Alternatively, in some implementations, the filter 213 is disposed within the chamber volume of the drip chamber 210. In some implementations, the filter 213 is an integral part of the drip chamber 210. During operation, fluid can flow through the inlet portion 212 of the drip chamber 210, through a filter 213 to an outlet portion 214 of the drip chamber 210. As medical fluid flows through the drip chamber 210, the fluid can be filtered prior to flowing out of the drip chamber 210 and through the second portion of tubing 206-b. As descried above in reference to FIG. 1, fluid within the chamber volume of the drip chamber 210 can pass through the filter 213 to prevent the transfer of bacteria, microorganisms, and/or other pathogens to the patient. As can be appreciated, a positive pressure differential can direct fluid flow through the filter 213. As described herein, the filter 213 can selectively filter the flow through the drip chamber 210. The filter 213 can have an average filter opening ranging between 15 to 200 microns. In some implementations, the average filter opening can range between 180 to 200 microns. Optionally, the filter 213 can have pores that vary in size. In some implementations, the filter 213 can be formed from a non-woven filter material.

During operation, the filter 213 can become clogged with particulate, limiting the filtering efficiency and flow through the filter 213. The conventional IV set 200 can include a hand pump 220 to dislodge sediment or particulate embedded in the filter 213, extending the life of the filter 213 and increasing the flow through the filter 213. The hand pump 220 can direct back flow or back pressure through the filter 213 to dislodge particulate embedded in the filter 266. In the depicted example, a clinician can actuate the hand pump 220 to create back flow through the filter 213.

FIGS. 3A-3C illustrate an infusion device having multiple pumping segments connected to a common fluid source, in accordance with some implementations. The multiple pumping segments (e.g., first and second deformable bodies 320-a and 320-b) of the infusion device 300 are configured to operate in parallel with each other to pump a fluid from the common fluid source. More specifically, the multiple pumping segments are configured to, when compressed according to respective timing patterns offset from each other, deliver a first portion of fluid from the fluid source from the first pumping segment to an IV set while filling the second pumping segment with a second portion of fluid from the fluid source from the fluid source, and to deliver the second portion of fluid from the fluid source from the second pumping segment to the IV set while the first pumping segment is filled from the fluid. In some implementations, the infusion device 300 includes one or more features described above in reference to FIG. 2. For example, the infusion device 300 includes one or more connectors 202, one or more clamps 204 and 208, tubing 206, a drip chamber 210, an injection site 230, and an outlet port 232. The infusion device 300 distributes medical fluid to a patient as described above in reference to FIG. 2. The infusion device 300 further includes at least two deformable bodies 320-a and 320-b to accelerate delivery of a fluid (i.e., increase flow of the fluid) to a patient 5 (FIG. 1) and reduce clinician (or other medical practitioner) fatigue, which allows for extended use of the infusion device 300.

In some implementations, each deformable body 320 of the at least two deformable bodies 320-a and 320-b is formed of a transparent material or a semi-transparent material to provide a visual indicator of the flow rate of fluid therethrough. Clinicians (or medical practitioners) can monitor and adjust the flow rate of the fluid based on the visual indicator provided by the at least two deformable bodies 320-a and 320-b. In some implementations, a first deformable body 320-a fluidically is coupled between the fluid source (e.g., container 36; FIG. 1) and an IV set or a portion thereof (e.g., one or more clamps 208, tubing 206, injection site 230, and/or outlet port 232) and a second deformable body 320-b is fluidically coupled between the fluid source and the IV set (or a portion thereof) in parallel with the first deformable body 320-a. In particular, the first deformable body 320-a is connected between a first union connector 311 configured to fluidly connect with an upstream infusion line (e.g., first portion of the tubing 206-a) for receiving the fluid from the fluid source and a second union connector 313, downstream from the first union connector 311, configured to connect to a downstream infusion line (e.g., second portion of the tubing 206-b) for providing the fluid to an IV set.

During operation, fluid can drip or otherwise flow through respective deformable body volumes 322-a and 322-b of the at least two deformable bodies 320-a and 320-b. Fluid can enter the at least two deformable bodies 320-a and 320-b through upper portions or inlet portions 324-a and 324-b defined by the respective deformable bodies 320-a and 320-b. The inlet portions 324-a and 324-b are in fluid communication with the first portion of the tubing 206-a. Fluid flow can exit the deformable body volumes 322-a and 322-b of the at least two deformable bodies 320-a and 320-b through respective lower portions or outlet portions 326-a and 326-b. When the respective deformable bodies 320-a and 320-b are not compressed, fluid, when allowed to flow, exits the deformable body volumes 322-a and 322-b at first and second default flow rates (e.g., flow rates unaccelerated by compression of the respective deformable bodies). The outlet portions 326-a and 326-b can be in fluid communication with a second portion of the tubing 206-b.

In some implementations, each deformable body 320 of the at least two deformable bodies 320-a and 320-b can be formed from a resilient or deformable material to allow the at least two deformable bodies 320-a and 320-b to be squeezed or compressed to draw in fluid for priming of an IV system. In addition, in some implementations, each deformable body 320 of the at least two deformable bodies 320-a and 320-b is actuated (e.g., squeezed or compressed) to accelerate fluid from the deformable body volumes 322-a and/or 322-b to the second portion of the tubing 206-b. For example, the first deformable body 320-a is configured to be hand-compressed by a user and, when compressed, direct a first portion of fluid from the fluid source from the first deformable body to an outlet of the IV set for administering the fluid to a patient at a first accelerated flow rate. Similarly, the second deformable body 320-b is configured to be hand-compressed by the user and, when compressed, direct a second portion of fluid from the fluid source from the second deformable body to the outlet of the IV set for administering the fluid to the patient at a second accelerated flow rate. As the skilled artisan will appreciate upon reading the descriptions provided herein, the first deformable body 320-a and the second deformable body 320-b allow for respective portions of fluid to be administered at first and second flow rates, respectively, without having to be compressed (e.g., when fluid is flowing from the fluid source). Compression of the first deformable body 320-a and the second deformable body 320-b allow for users to adjust the flow rate of the fluid as needed. In some implementations, the resilient or deformable material includes silicone, rubber, thermoplastic elastomers, plastics, polyurethane, and/or other flexible material that can be squeezed and return to its resting state.

In some implementations, the at least two deformable bodies 320-a and 320-b equalize pressure differentials between the deformable body volumes 322-a and 322-b and the environment during operation. In some implementations, by reducing the deformable body volumes 322-a and/or 322-b, the fluid within the deformable body volumes 322-a and/or 322-b is pressurized. The pressurized fluid within the deformable body volumes 322-a and/or 322-b can be accelerated to distribute the fluid to the user at a faster rate. In some implementations, the at least two deformable bodies 320-a and 320-b draw in fluid for priming the IV system. For example, the deformable body volumes 322-a and 322-b can be filled with a desired volume of fluid during the priming operation. Advantageously, due to the deformable body volumes 322-a and/or 322-b, the at least two deformable bodies 320-a and 320-b improve flow through the infusion device 300 rapidly while reducing the strain and fatigue on clinicians (or medical practitioners).

As shown in FIGS. 3B and 3C show the compression of the at least two deformable bodies 320-a and 320-b. In particular, FIG. 3B shows compression of a first deformable body 320-a and FIG. 3C shows compression of a second deformable body 320-a. In some implementations, the at least two deformable bodies 320-a and 320-b are simultaneously compressed. Alternatively, in some implementations, the at least two deformable bodies 320-a and 320-b are compressed in an alternating sequence (e.g., compression of the first deformable body 320-a before the second deformable body 320-a and vice versa). In some implementations, a flow rate exiting each deformable body of the at least two deformable bodies 320-a and 320-b is the same (e.g., when compressed or without any compression force being applied). Alternatively, in some implementations, a flow rate exiting each deformable body of the at least two deformable bodies 320-a and 320-b is distinct. In some implementations, the flow rate exiting each deformable body of the at least two deformable bodies 320-a and 320-b is based, at least in part, on one or more of the material of the at least two deformable bodies 320-a and 320-b, a compression force applied by a user, amount of fluid in the deformable body volumes 322-a and 322-b, pressure within the at least two deformable bodies 320-a and 320-b, and/or other factors.

In some implementations, each deformable body 320 of the at least two deformable bodies 320-a and 320-b includes a filter analogous to filter 213 described above in reference to FIG. 2. More specifically, similar to the drip chamber 210, filters of the at least two deformable bodies 320-a and 320-b allow for filtration of fluid passing therethrough. In some implementations, the at least two deformable bodies 320-a and 320-b integrate a filter. In some implementations, due to the increased size and volumes of the at least two deformable bodies 320-a and 320-b, the filters disposed within the at least two deformable bodies 320-a and 320-b are larger and have more surface area than the filter 213 utilized within the drip chamber 210. In some implementations, the filter can be any suitable size. In some implementations, the at least two deformable bodies 320-a and 320-b including filters can be used in place of the filter 213 of drip chamber 210. Alternatively, in some implementations, the at least two deformable bodies 320-a and 320-b including filters can be used in place of the drip chamber 210 (i.e., removing an additional component of the IV system).

In some implementations, during operation, as fluid flows through the at least two deformable bodies 320-a and 320-b, the fluid is filtered prior to flowing out of the at least two deformable bodies 320-a and 320-b and into the second portion of the tubing 206-b. As described above, the filter to prevents the transfer of bacteria, microorganisms, and/or other pathogens to the patient. In some implementations, the filter has an average filter opening ranging between 15 to 200 microns. In some implementations, the average filter opening ranges between 180 to 200 microns. Optionally, the filter can have pores that vary in size. In some implementations, the filter can be formed from a non-woven filter material.

In addition to accelerating a flow of fluid, the pressurized fluid within the deformable body volumes 322-a and/or 322-b is such that, upon actuation or compression of a deformable body of the at least two deformable bodies 320-a and 320-b, back flow is forced through filters of the at least two deformable bodies 320-a and 320-b (e.g., analogous to filter 213 described above in reference to FIG. 2), displacing or dislodging particulate from the filter. Advantageously, due to the deformable body volumes 322-a and/or 322-b, the at least two deformable bodies 320-a and 320-b improve flow through the infusion device 300 rapidly while reducing the strain and fatigue on clinicians (or medical practitioners).

FIGS. 4A-4C illustrate another infusion device having multiple pumping segments connected to a common fluid source, in accordance with some implementations. The multiple pumping segments (e.g., first and second deformable bulb bodies 405-a and 405-b coupled between first and second union connectors 311 and 313) of the infusion device 400 are configured to operate in parallel with each other to pump a fluid from the common fluid source. Infusion device 400 includes one or more features described above in reference to FIGS. 3A-3C. For example, the infusion device 400 includes one or more connectors 202, one or more clamps 204 and 208, tubing 206, a drip chamber 210, an injection site 230, and an outlet port 232. The infusion device 400 distributes medical fluid to a patient as described above in reference to FIGS. 3A-3C. The infusion device 400 further includes at least two deformable bulb bodies 405-a and 405-b to accelerate delivery of a fluid (i.e., increase flow of the fluid) to a patient 5 (FIG. 1) and reduce clinician (or other medical practitioner) fatigue, which allows for extended use of the infusion device 400. In some implementations, each bulb body of the at least two deformable bulb bodies 405-a and 405-b includes an inlet portion 410 that is in fluid communication with the first portion of the tubing 206-a, deformable bulb body volumes 430, and outlet portions 440 in fluid communication with the second portion of the tubing 206-b. In some implementations, a first deformable bulb body 405-a fluidically is coupled between the fluid source (e.g., container 36; FIG. 1) and an IV set or a portion thereof (e.g., one or more clamps 208, tubing 206, injection site 230, and/or outlet port 232) and a second deformable bulb body 405-b is fluidically coupled between the fluid source and the IV set (or a portion thereof) in parallel with the first deformable bulb body 405-a.

In some implementations, the at least two deformable bulb bodies 405-a and 405-b include respective filters 420. In some implementations, the least two deformable bulb bodies 405-a and 405-b including filters can be used in place of the drip chamber 210. The at least two deformable bulb bodies 405-a and 405-b are configured to perform similar functions as the at least two deformable body 220-a and 220-b described in reference to FIGS. 3A-3C.

Each bulb body of the least two deformable bulb bodies 405-a and 405-b can generate higher flow rates when compressed or actuated (relative to hand pump 220; FIG. 2). Further, each bulb body of the at least two deformable bulb bodies 405-a and 405-b has an ergonomic shape that further reduce clinician fatigue further allowing for extended periods of use.

FIGS. 4B and 4C show the compression of the at least two deformable bulb bodies 405-a and 405-b. In particular, FIG. 4B shows compression of a first deformable bulb body 405-a and FIG. 4C shows compression of a second deformable bulb body 405-b. In some implementations, the at least two deformable bulb bodies 405-a and 405-b are simultaneously compressed. Alternatively, in some implementations, the at least two deformable bulb bodies 405-a and 405-b are compressed in an alternating sequence (e.g., compression of the first deformable bulb body 405-a before the second bulb body pump 405-a and vice versa). In some implementations, a flow rate exiting each bulb body pump of the at least two deformable bulb bodies 405-a and 405-b is the same (e.g., when compressed or without any compression force being applied). Alternatively, in some implementations, a flow rate exiting each bulb body pump of the at least two deformable bulb bodies 405-a and 405-b is distinct. In some implementations, the flow rate exiting each deformable bulb body of the at least two deformable bulb bodies 405-a and 405-b is based, at least in part, on one or more of the material of the at least two deformable bulb bodies 405-a and 405-b, a compression force applied by a user, amount of fluid in the deformable bulb body volumes 430, pressure within the at least two deformable bulb bodies 405-a and 405-b, and/or other factors.

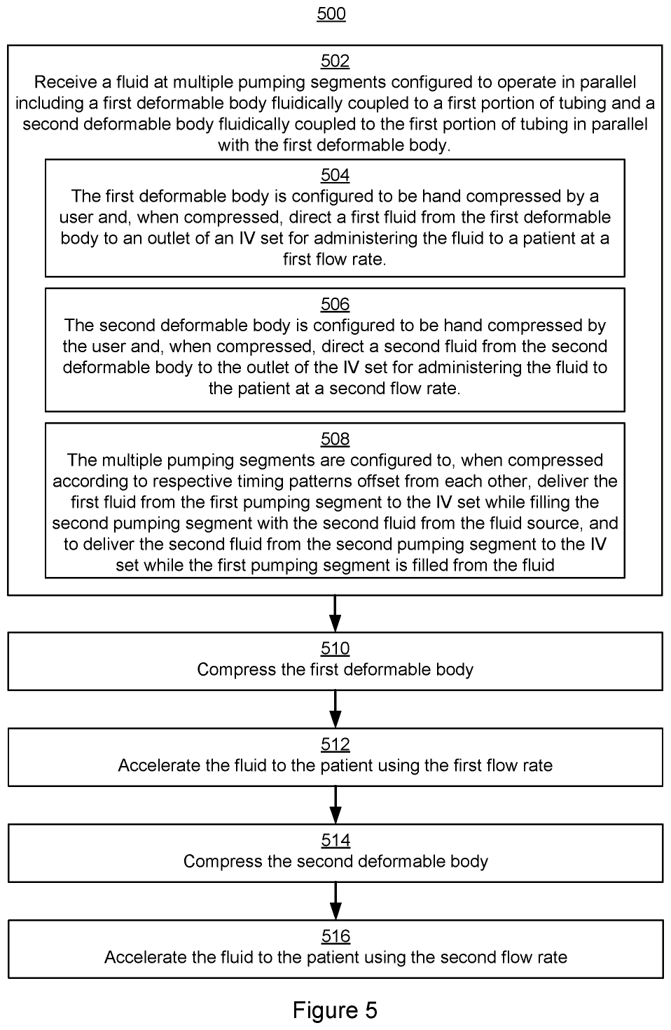

FIG. 5 is a flowchart illustrating a method 500 of forming an apparatus for accelerating a fluid from a fluid source to a patient, in accordance with some implementations. Method 500 can be used to form an infusion device including multiple pumping segments as described above in reference to FIGS. 3A-4C. Methods consistent with the present disclosure may include at least some, but not all, of the operations illustrated in method 500, performed in a different sequence. Furthermore, methods consistent with the present disclosure may include at least two or more steps as in method 500 performed overlapping in time, or almost simultaneously.

Method 500 includes connecting (502), between a first union connector 311 (FIGS. 3A-4C) configured to fluidly connect with an upstream infusion line (e.g., first portion of the tubing 206-a) for receiving the fluid from the fluid source and a second union connector 313 (FIGS. 3A-4C), downstream from the first union connector 311, configured to connect to a downstream infusion line (e.g., second portion of the tubing 206-b) for providing the fluid to an IV set, a first deformable body (e.g., first deformable body 320-a or deformable bulb body 405-a; FIGS. 3A-4C) configured to be hand-compressed by a user. The first deformable body, when compressed, is configured to direct a first portion of the fluid from the first deformable body to the IV set for administering the first portion of the fluid to the patient at a first accelerated flow rate. The method further includes connecting, between the first and second union connectors 311 and 313, a second deformable body (e.g., second deformable body 320-b or deformable bulb body 405-b; FIGS. 3A-4C) in parallel with the first deformable body. The second deformable body is configured to be hand-compressed by the user and, when compressed, direct a second portion of the fluid from the second deformable body to the IV set for administering the fluid to the patient at a second accelerated flow rate. Additional examples of the first and second deformable bodies are provided above in reference to FIGS. 3A-4C.

The first and second deformable bodies are configured to, when compressed according to respective timing patterns offset from each other, operate (530) together to deliver fluid from the fluid source, such that the fluid is delivered from the fluid source to the patient at a flow rate greater than the first and second default flow rates and a default flow rate of the fluid source. In other words, compression of the first and/or second deformable bodies causes fluid from the fluid source to flow at a rate faster than when the deformable bodies are not compressed. Delivering fluid from the fluid source includes delivering (540) the first portion of the fluid from the first pumping segment (e.g., the first deformable body) to the IV set while filling the second pumping segment (e.g., the second deformable body) with the second portion of the fluid from the fluid source and delivering (550) the second portion of the fluid from the second pumping segment to the IV set while the first pumping segment is filled with the first portion of the fluid from the fluid. In some implementations, method 500 includes connecting, a drip chamber 210 (e.g., FIGS. 3A-4C), upstream of the first union connector 311, that is configured to provide a visual indicator of the default flow rate of the fluid source.

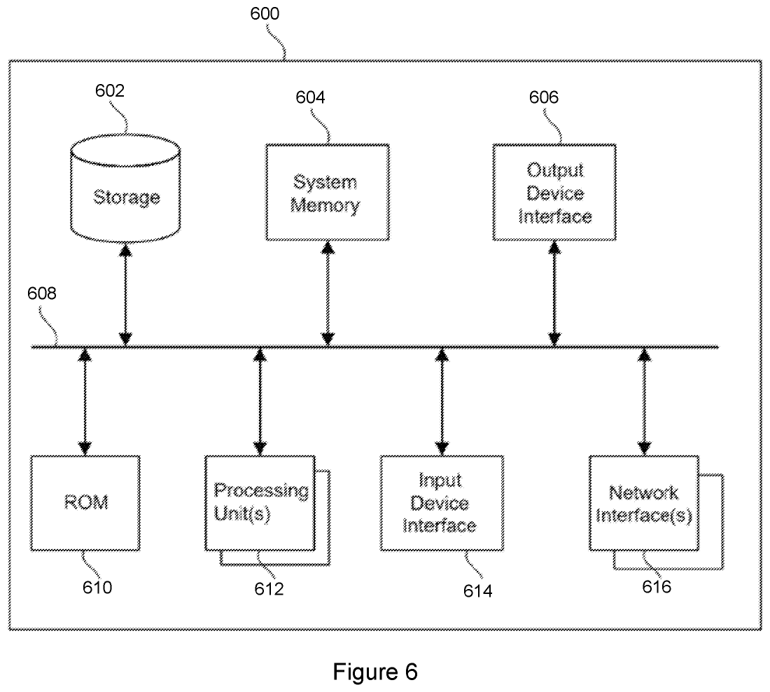

FIG. 6 is a conceptual diagram illustrating an example electronic system 600 for controlling a pump, according to aspects of the subject technology. Electronic system 600 may be a specifically configured computing device for execution of software associated with components and processes provided by FIGS. 1 through 5, including but not limited to controller 32 of IV pump 30. Electronic system 600 may be representative, in combination with the disclosure regarding FIGS. 1 through 5.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as a random-access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations. Such storage and/or memory devices 602, 604 may be representative of memory of controller 32.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”), such as those shown in controller 32 of FIG. 1. Output device interfaces 606 (e.g., shown as a display in the controller 60 of FIG. 1) enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user, e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An infusion device having multiple pumping segments connected to a common fluid source and configured to operate in parallel with each other to pump a fluid from the common fluid source. The multiple pumping segments including a first deformable body fluidically coupled between the fluid source and an IV set, the first deformable body configured to be hand-compressed by a user and, when compressed, direct a first portion of fluid from the fluid source from the first deformable body to an outlet of the IV set for administering the fluid to a patient at a first flow rate. The multiple pumping segments including a second deformable body fluidically coupled between the fluid source and the IV set in parallel with the first deformable body, the second deformable body configured to be hand-compressed by the user and, when compressed, direct a second portion of fluid from the fluid source from the second deformable body to the outlet of the IV set for administering the fluid to the patient at a second flow rate. The multiple pumping segments are configured to, when compressed according to respective timing patterns offset from each other, deliver the first portion of fluid from the fluid source from the first deformable body to the IV set while filling the second deformable body with the second portion of fluid from the fluid source from the fluid source, and to deliver the second portion of fluid from the fluid source from the second deformable body to the IV set while the first deformable body is filled from the fluid.
Clause 2. The infusion device of Clause 1, the first and/or second deformable bodies are simultaneously compressed.
Clause 3. The infusion device of Clause 1, the first and/or second deformable bodies are compressed in an alternating sequence.
Clause 4. The infusion device of any of Clause 1 through Clause 3, the first and second flow rates are the same.
Clause 5. The infusion device of any of Clause 1 through Clause 4, the infusion device further including one or more filters fluidically coupled to the first and/or second deformable bodies. The one or more filters capture particulate from the fluid directed from the first and/or second deformable bodies.
Clause 6. The infusion device of Clause 5, the first and/or second deformable bodies, when compressed, direct back flow from the first and/or second deformable bodies through the one or more filters, agitating particulate captured in the filter.
Clause 7. The infusion device of any of Clause 5 and Clause 6, the one or more filters are disposed adjacent to first and/or second inlets of the first and second deformable bodies, respectively.
Clause 8. The infusion device of any of Clause 5 and Clause 6, the one or more filters are disposed within first and/or second inlets of the first and second deformable bodies, respectively.
Clause 9. The infusion device of any of Clause 1 through Clause 8, the first and second deformable bodies comprise a resilient material such that the first and second deformable bodies rebound to an uncompressed state.
Clause 10. The infusion device of any of Clause 1 through Clause 9, the first and second deformable bodies comprise a transparent material or a semi-transparent material.
Clause 11. The infusion device of any of Clause 1 through Clause 10, the first and second deformable bodies comprise a substantially cylindrical shape.
Clause 12. The infusion device of any of Clause 1 through Clause 11, the first deformable body is fluidically coupled between the fluid source and the IV set via a first inlet and the second deformable body is fluidically coupled between the fluid source and the IV set via a second inlet.
Clause 13. An infusion system having multiple pumping segments connected to a common fluid source and configured to operate in parallel with each other to pump a fluid from the common fluid source. The infusion system including a first portion of tubing, a second portion of tubing, and multiple pumping segments configured to operate in parallel. The multiple pumping segments including a first deformable body fluidically coupled to the first portion of tubing, the first deformable body configured to be hand-compressed by a user and, when compressed, direct a first portion of fluid from the fluid source from the first deformable body to an outlet fluidically coupled to the second portion of tubing for administering the fluid to a patient at a first flow rate. The multiple pumping segments further including a second deformable body fluidically coupled to the first portion of tubing in parallel with the first deformable body, the second deformable body configured to be hand-compressed by the user and, when compressed, direct a second portion of fluid from the fluid source from the second deformable body to the outlet fluidically coupled to the second portion of tubing for administering the fluid to the patient at a second flow rate. The multiple pumping segments are configured to, when compressed according to respective timing patterns offset from each other, deliver the first portion of fluid from the fluid source from the first deformable body to an IV set while filling the second deformable body with the second portion of fluid from the fluid source from the fluid source, and to deliver the second portion of fluid from the fluid source from the second deformable body to the IV set while the first deformable body is filled from the fluid.
Clause 14. The infusion system of Clause 13, the first and/or second deformable bodies are simultaneously compressed.
Clause 15. The infusion system of Clause 13, the first and/or second deformable bodies are compressed in an alternating sequence.
Clause 16. The infusion system of any of Clause 13 through Clause 15, the first and second flow rates are the same.
Clause 17. The infusion system of any of Clause 13 through Clause 16, the infusion system further includes one or more filters fluidically coupled to the first and/or second deformable bodies. The one or more filters capture particulate from the fluid directed from the first and/or second deformable bodies.
Clause 18. The infusion system of any of Clause 13 through Clause 17, the first and second deformable bodies comprise a resilient material such that the first and second deformable bodies rebound to an uncompressed state.
Clause 19. A method of forming an apparatus for accelerating a fluid from a fluid source to a patient, the method including connecting, between a first union connector configured to fluidly connect with an upstream infusion line for receiving the fluid from the fluid source and a second union connector, downstream from the first union connector, configured to connect to a downstream infusion line for providing the fluid to an IV set, a first deformable body configured to be hand-compressed by a user and, when compressed, direct a first portion of the fluid from the first deformable body to the IV set for administering the first portion of the fluid to the patient at a first accelerated flow rate. The method further includes connecting, between the first and second union connectors, a second deformable body in parallel with the first deformable body, the second deformable body configured to be hand-compressed by the user and, when compressed, direct a second portion of the fluid from the second deformable body to the IV set for administering the fluid to the patient at a second accelerated flow rate. The first and second deformable bodies are configured to, when compressed according to respective timing patterns offset from each other, operate together to deliver fluid from the fluid source by delivering the first portion of the fluid from the first deformable body to the IV set while filling the second deformable body with the second portion of the fluid from the fluid source, and delivering the second portion of the fluid from the second deformable body to the IV set while the first deformable body is filled with the first portion of the fluid from the fluid, such that the fluid is delivered from the fluid source to the patient at a flow rate greater than first and second default flow rates and a default flow rate of the fluid source.

### Further Consideration:

In some implementations, any of the clauses herein may depend from any one of the independent clauses or any one of the dependent clauses. In one aspect, any of the clauses (e.g., dependent, or independent clauses) may be combined with any other one or more clauses (e.g., dependent, or independent clauses). In one aspect, a claim may include some or all of the words (e.g., steps, operations, means or components) recited in a clause, a sentence, a phrase, or a paragraph. In one aspect, a claim may include some or all of the words recited in one or more clauses, sentences, phrases, or paragraphs. In one aspect, some of the words in each of the clauses, sentences, phrases, or paragraphs may be removed. In one aspect, additional words or elements may be added to a clause, a sentence, a phrase, or a paragraph. In one aspect, the subject technology may be implemented without utilizing some of the components, elements, functions, or operations described herein. In one aspect, the subject technology may be implemented utilizing additional components, elements, functions, or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It will be understood that, although the terms “first,” “second,” etc. may be used herein to describe various elements, these elements should not be limited by these terms. These terms are only used to distinguish one element from another. Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The terminology used herein is for the purpose of describing particular implementations only and is not intended to be limiting of the claims. As used in the description of the implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An infusion device having multiple pumping segments connected to a common fluid source and configured to operate in parallel with each other to pump a fluid from the common fluid source, comprising:
a first deformable body fluidic ally coupled between the fluid source and an IV set, the first deformable body configured to be hand-compressed by a user and, when compressed, direct a first portion of fluid from the fluid source from the first deformable body to an outlet of the IV set for administering the fluid to a patient at a first flow rate; and
a second deformable body fluidic ally coupled between the fluid source and the IV set in parallel with the first deformable body, the second deformable body configured to be hand-compressed by the user and, when compressed, direct a second portion of fluid from the fluid source from the second deformable body to the outlet of the IV set for administering the fluid to the patient at a second flow rate,
wherein the multiple pumping segments are configured to, when compressed according to respective timing patterns offset from each other, deliver the first portion of fluid from the fluid source from the first deformable body to the IV set while filling the second deformable body with the second portion of fluid from the fluid source from the fluid source, and to deliver the second portion of fluid from the fluid source from the second deformable body to the IV set while the first deformable body is filled from the fluid.

2. The infusion device of claim 1, wherein the first deformable body and/or the second deformable body are simultaneously compressed.

3. The infusion device of claim 1, wherein the first deformable body and/or the second deformable body are compressed in an alternating sequence.

4. The infusion device of claim 1, wherein the first and second flow rates are the same.

5. The infusion device of claim 1, further comprising:
one or more filters fluidically coupled to the first deformable body and/or the second deformable body, wherein the one or more filters capture particulate from the fluid directed from the first deformable body and/or the second deformable body.

6. The infusion device of claim 5, wherein the first deformable body and/or the second deformable body, when compressed, direct back flow from the first deformable body and/or the second deformable body through the one or more filters, agitating particulate captured in the filter.

7. The infusion device of claim 5, wherein the one or more filters are disposed adjacent to first and/or second inlets of the first and second deformable bodies, respectively.

8. The infusion device of claim 5, wherein the one or more filters are disposed within first and/or second inlets of the first and second deformable bodies, respectively.

9. The infusion device of claim 1, wherein the first and second deformable bodies comprise a resilient material such that the first and second deformable bodies rebound to an uncompressed state.

10. The infusion device of claim 1, wherein the first and second deformable bodies comprise a transparent material or a semi-transparent material.

11. The infusion device of claim 1, wherein the first and second deformable bodies comprise a substantially cylindrical shape.

12. The infusion device of claim 1, wherein the first deformable body is fluidically coupled between the fluid source and the IV set via a first inlet and the second deformable body is fluidically coupled between the fluid source and the IV set via a second inlet.

13. An infusion system having multiple pumping segments connected to a common fluid source and configured to operate in parallel with each other to pump a fluid from the common fluid source, comprising:
a first portion of tubing;
a second portion of tubing; and
multiple pumping segments configured to operate in parallel and comprising:
a first deformable body fluidically coupled to the first portion of tubing, the first deformable body configured to be hand-compressed by a user and, when compressed, direct a first portion of fluid from the fluid source from the first deformable body to an outlet fluidically coupled to the second portion of tubing for administering the fluid to a patient at a first flow rate; and
a second deformable body fluidically coupled to the first portion of tubing in parallel with the first deformable body, the second deformable body configured to be hand-compressed by the user and, when compressed, direct a second portion of fluid from the fluid source from the second deformable body to the outlet fluidically coupled to the second portion of tubing for administering the fluid to the patient at a second flow rate,
wherein the multiple pumping segments are configured to, when compressed according to respective timing patterns offset from each other, deliver the first portion of fluid from the fluid source from the first deformable body to an IV set while filling the second deformable body with the second portion of fluid from the fluid source from the fluid source, and to deliver the second portion of fluid from the fluid source from the second deformable body to the IV set while the first deformable body is filled from the fluid.

14. The infusion system of claim 13, wherein the first deformable body and/or the second deformable body are simultaneously compressed.

15. The infusion system of claim 13, wherein the first deformable body and/or the second deformable body are compressed in an alternating sequence.

16. The infusion system of claim 13, wherein the first and second flow rates are the same.

17. The infusion system of claim 13, further comprising:
one or more filters fluidically coupled to the first deformable body and/or the second deformable body, wherein the one or more filters capture particulate from the fluid directed from the first deformable body and/or the second deformable body.

18. The infusion system of claim 13, wherein the first and second deformable bodies comprise a resilient material such that the first and second deformable bodies rebound to an uncompressed state.

19. A method of forming an apparatus for accelerating a fluid from a fluid source to a patient, comprising:
connecting, between a first union connector configured to fluidly connect with an upstream infusion line for receiving the fluid from the fluid source and a second union connector, downstream from the first union connector, configured to connect to a downstream infusion line for providing the fluid to an IV set, a first deformable body configured to be hand-compressed by a user and, when compressed, direct a first portion of the fluid from the first deformable body to the IV set for administering the first portion of the fluid to the patient at a first accelerated flow rate; and
connecting, between the first and second union connectors, a second deformable body in parallel with the first deformable body, the second deformable body configured to be hand-compressed by the user and, when compressed, direct a second portion of the fluid from the second deformable body to the IV set for administering the fluid to the patient at a second accelerated flow rate,
wherein the first and second deformable bodies are configured to, when compressed according to respective timing patterns offset from each other, operate together to deliver fluid from the fluid source by delivering the first portion of the fluid from the first deformable body to the IV set while filling the second deformable body with the second portion of the fluid from the fluid source, and delivering the second portion of the fluid from the second deformable body to the IV set while the first deformable body is filled with the first portion of the fluid from the fluid, such that the fluid is delivered from the fluid source to the patient at a flow rate greater than first and second default flow rates and a default flow rate of the fluid source.

20. The method of claim 19, further comprising:
connecting, a drip chamber upstream of the first union connector that is configured to provide a visual indicator of the default flow rate of the fluid source.
